(19) United States
(12) Patent Application Publication
(10) Pub. No.: US 2008/0175829 A1
(43) Pub. Date: Jul. 24, 2008

(54) METHOD FOR INDUCING NEURAL DIFFERENTIATION

(75) Inventors: Henrich CHENG, Taipei (TW); Shun-Fen TZENG, Tainan (TW)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: Henrich CHENG, Taipei (TW)

(21) Appl. No.: 12/015,367

(22) Filed: Jan. 16, 2008

Related U.S. Application Data
(63) Continuation-in-part of application No. 10/873,640, filed on Jun. 23, 2004.

(51) Int. Cl. A61K 48/00; C12N 9/00

(52) U.S. Cl. 424/93.21; 435/183

(57) ABSTRACT

A method for inducing neural differentiation includes treating a bone marrow stem cell with a neurotrophic factor and/or dibutyryl cAMP (dbcAMP), wherein the neurotrophic factor includes or is glial cell line-derived neurotrophic factor (GDNF) or pituitary adenylate cyclase-activating polypeptide (PACAP).

### CROSS-REFERENCE TO RELATED APPLICATIONS

This application is a continuation-in-part of copending U.S. patent application Ser. No. 10/873,640, filed Jun. 23, 2004, the disclosure of which is hereby incorporated herein by reference in its entirety.

### BACKGROUND OF THE INVENTION

1. Field of the Invention

The invention mainly relates to a method for inducing neural differentiation with no toxicity.

2. Description of the Related Art

Loss of neural cells after injury of the central nervous system (CNS) makes CNS repair difficult. Many studies of show that neural stem cells (NSCs) isolated from various rodent and human CNS areas are capable of differentiating into neural cells in the adult rodent CNS under the influence of the environment and/or exogenous growth factors (F. H. Gage, Mammalian neural stem cells. Science. 287 (2000) 1433-1438; J. Price, B. P. Williams, Neural stem cells, Curr. Opin. Neurobiol. 11 (2001) 564-567). Thus, replenishment of NSCs is thought to be a potential strategy for human CNS treatment (D. A. Peterson, Stem cells in brain plasticity and repair, Curr. Opin. Pharmacol. 2 (2002) 34-42).

Stem cells derived from human bone marrow (hBMSCs) are heterogeneous in morphology. They multipotentially differentiate into osteoblasts, adipocytes, chondrocytes and muscle and can also generate neurons (E. Mezey, K. J. Chandross, G. Harta, R. A. Maki, S. R. McKercher, Turning Blood into Brain: Cells Bearing Neuronal Antigens Generated in vivo from Bone Marrow. Science, 290 (2000) 1779-1782; E. Mezey, S. Key, G. Vogelsang, I. Szalayova, G. D. Lange, B. Crain, Transplanted bone marrow generates new neurons in human brains, Proc. Natl. Acad. Sci. USA. 100 (2003) 1364-1369; Sanchez-Ramos et al. (2000); D. Woodbury, E. J. Schwarz, D. J. Prockop, I. B. Black, Adult rat and human bone marrow stromal cells differentiate into neurons, J. Neurosci. Res. 61 (2000) 364-370). Recently, human and mouse BMSCs have been reported to express neuronal progenitor marker (nestin), neuron-specific nuclear protein (NeuN), and glial acidic fibrillary protein (GFAP) after stimulation with retinoic acid and epidermal growth factor (EGF) or brain-derived neurotrophic factor (BDNF) (Sanchez-Ramos et al. (2000)). It has also been demonstrated that transplanted BMSCs are able to differentiate between the neuronal and glial lineages in damaged CNS (J. R. Sanchez-Ramos. Neural cells derived from adult bone marrow and umbilical cord blood. J. Neurosci Res. 69 (2002) 880-893). Moreover, it is found in Chopp et al. that the transplantation of BMSCs can improve functional recovery in rats with focal cerebral ischemia (J. Chen, Y. Li, M. Chopp. Intracerebral transplantation of bone marrow with BDNF after MCAo in rat. Neuropharmacology. 39 (2000) 711-716), in rats with traumatic brain injury (D. Lu, Y. Li, L. Wang, J. Chen, A. Mahmood, M. Chopp. Intraarterial administration of marrow stromal cells in a rat model of traumatic brain injury. J Neurotrauma. 18 (2001) 813-819), and in mice with Parkinson's disease (Y. Li, J. Chen, L. Wang, L. Zhang, M. Lu, M. Chopp. Intracerebral transplantation of bone marrow stromal cells in a 1-methyl-4-phenyl-1,2,3,6-tetrahydropyridine mouse model of Parkinson's disease. Neurosci Lett. 316 (2001) 67-70). These findings indicate the potential role as a useful cell source of CNS cell therapy in humans.

Nevertheless, the isolation of hBMSCs by adherence to the culture petri dish primarily generates heterogeneous populations (A. J. Friedenstein, J. E. Gorskaja, N. N. Kulagina. Fibroblast precursors in normal and irradiated mouse hematopoietic organs. Exp Hematol. 4 (1976) 267-274). Therefore, several methods have been developed based on their different sizes and specific surface markers to generate homogeneous populations using cell sorting (S. C. Hung, N. J. Chen, S. L. Hsieh, H. Li, H. L. Ma, W. H. Lo, Isolation and characterization of size-sieved stem cells from human bone marrow, Stem Cells. 20 (2002) 249-258; R. Zohar, J. Sodek, C. A. McCulloch, Characterization of stromal progenitor cells enriched by flow cytometry, Blood. 90 (1997) 3471-3481). Accordingly, Hung et al. (2002) have recently developed an efficient isolation of the homogeneous population from human bone marrow on the basis of cell size and adherent capacity via using Percoll gradient separation and a 3-μm porous sieve to dispose of smaller cells. The purified hBMSC population that was generated has been referred to as size-sieved cells (SSCs), and they have a greater renewal capability than heterogeneous populations of hBMSCs (Hung et al. (2002)). SSCs lack the surface markers of the early hematopoietic stem cells, CD34 and AC133, at the passage 2 to 3, and fail to express markers for osteogenic MSCs and mature osteogenic precursors (Hung et al. (2002)). However, these cells express Thy-1, matrix receptors (CD44 and CD105), and integrins (CD29 and CD51). SSCs are multipotential, and can rise the osteogenic, adipogenic, and chondrogenic lineages under the influence of environmental signaling (Hung et al. (2002)). SSCs have also been found to generate neural cells electrically with the stimulation of antioxidant agents such as β-mercaptoethanol and retinoic acid, which are often used in vitro to induce the neural differentiation of stem cells (S. C. Hung, H. Cheng, C. Y. Pan, M. J. Tsai, L. S. Kao, H. L. Ma, In vitro differentiation of size-sieved stem cells into electrically active neural cells, Stem Cells. 20 (2002) 522-529). Although β-mercaptoethanol and retinoic acid have a potent effect on the differentiation of SSCs into functional neurons, the role of the two factors in CNS tissue repair remains to be defined.

However, stimulating SSCs to generate neural cells with antioxidant agents such as β-mercaptoethanol and retinoic acid can only be applied in vitro. β-Mercaptoethanol is a toxic reagent. Moreover, retinoic acid is a carcinogen. Both of the antioxidant agents cause damage to an animal and transplanting the stimulated neural cells leads to the receiver's death.

### BRIEF SUMMARY OF THE INVENTION

The invention provides a novel method for morphological transformation of SSCs from fibroblastic-like shapes to process-bearing forms with neurotrophic factors which are safe and effective in stimulating the changes of neural cell morphology. Furthermore, the neural cells obtained according to the invention are suitable for repairing neural defects in animals.

One subject of the invention is to provide a method for inducing neural differentiation comprising treating a bone marrow stem cell with a neurotrophic factor and/or dibutyryl cAMP (dbcAMP), wherein the neurotrophic factor comprises glial cell line-derived neurotrophic factor (GDNF) or pituitary adenylate cyclase-activating polypeptide (PACAP).

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 illustrates phase contrast images of neuronal-like transformation of SSCs. It indicates that SSCs become process-bearing cells in insulin-transferrin-selenium (ITS) medium (described hereinafter) alone (0), and in ITS medium with 50 ng/ml GDNF or 20 ng/ml PACAP (magnification=200×).

FIG. 2 illustrates phase contrast images of neuronal-like transformation of SSCs by GDNF, PACAP, and dbcAMP. It indicates that the morphology of SSCs in serum containing medium is fibroblastic-like, and becomes a process-bearing form in serum withdrawal medium (ITS medium alone; 0). Furthermore, the processes of SSCs treated for 7 days in ITS medium with GDNF (50 ng/ml), PACAP (20 ng/ml), dbcAMP (0.1 mM), GDNF (50 ng/ml)+PACAP (20 ng/ml), or GDNF (50 ng/ml)+dbcAMP (0.1 mM) are elongated and highly branching (magnification=200×).

FIG. 3 illustrates the results of neuronal specific markers NF-L and NF-H expression wherein Western Blotting analysis shows that neuronal specific marker NF-L is upregulated in SSCs 7 days after treatment with GDNF (50 ng/ml) or PACAP (10 and 20 ng/ml) in ITS medium when compared to that observed in ITS medium alone (0).

FIG. 4 illustrates the results of NF-L and α-tubulin expressions, wherein Western Blotting analysis shows that neuronal specific marker NF-L is upregulated in SSCs 7 days after treatments indicated as above. Moreover, the level of neuronal nonspecific cytoskeleton protein α-tubulin in SSCs is increased by various treatments indicated as above. Treatment with ITS-medium alone is represented as 0.

FIG. 5 illustrates the results of Immunofluorescence staining for α-internexin in SSCs wherein SSCs are treated for 7 days in ITS medium alone (0) or with GDNF (50 ng/ml), PACAP (20 ng/ml), and dbcAMP (0.1 mM). The cultures are then subjected to immunofluorescence staining for α-internexin. Branching (arrows) and elongation (arrowheads) of the processes in SSCs treated with GDNF and dbcAMP are more extensive over the ITS medium alone (0). Scale bar=50 μm.

FIG. 6 illustrates images of neuronal-like transformation of SSCs. It indicate that the vesicle protein, synapsin-1, is already expressed when SSCs are cultured for 7 days in ITS medium alone (0). Moreover, treatment with GDNF (50 ng/ml) or PACAP (20 ng/ml) in ITS medium for 7 days induces further elongation (arrowheads) and increases branching of the processes (arrows). Scale bar=50 μm.

FIG. 7 is a diagram of the location of induced spinal cord injury in a study of hindlimb locomotor recovery after SSCs transplantation in rats.

FIG. 8 comprises FIGS. 8A-8C and shows immunostaining for the specific marker (Hu) for SSCs post-transplantation relating to the study diagrammed in FIG. 7. FIG. 8A is an image 24 hours post-transplantation. FIG. 8B is an image 8 days post-transplantation. FIG. 8C shows what location of the transplantations relative to the epicenter.

FIG. 9 is a graph showing the locomotor results of the study of FIGS. 7 and 8.

### DETAILED DESCRIPTION OF THE INVENTION

The invention is to provide a method for inducing neural differentiation comprising treating a bone marrow stem cell with a neurotrophic factor and/or dibutyryl cAMP (dbcAMP), wherein the neurotrophic factor comprises glial cell line-derived neurotrophic factor (GDNF) or pituitary adenylate cyclase-activating polypeptide (PACAP).

According to the invention, the bone marrow stem cell is taken for preparing functional neural cells. Stem cells derived from human bone marrow have the potential of differentiating into neurons and are considered to be the best material for regenerating neural cells. Preferably, the bone marrow stem cell is a size-sieved stem cell derived from human bone marrow. Size-sieved stem cells are developed based on their different sizes and specific surface markers to generate homogeneous populations by using cell sorting to avoid heterogeneous population generation of primary bone marrow stem cells cultures. Furthermore, the size-sieved stem cells have greater renewal capability than heterogeneous populations. The size-sieved stem cell derived from human bone marrow is sieved more preferably with a 3-μm porous sieve. The size-sieved stem cells are efficiently isolated as the homogeneous population from human bone marrow on the basis of cell size and adherent capacity via using Percoll gradient separation and a porous sieve to dispose of smaller cells.

According to the invention, the neurotrophic factor is taken as a stimulus to induce neural differentiation. In view of the neurotrophic factor being a microenvironmental factor that exists in a normal physiological condition with less damage than that of a chemical reagent, it is regarded as a safe reagent to treat the bone marrow stem cell for the purpose of transplantation into an animal. According to the invention, the neurotrophic factor comprises glial cell line-derived neurotrophic factor or pituitary adenylate cyclase-activating polypeptide.

Glial cell line-derived neurotrophic factor (GDNF), which is a potent survival factor for many CNS neuronal cell populations, has the therapeutic potential for various CNS disorders. The therapeutic value of GDNF has been recently reviewed by Airaksinen and Saarma (M. S. Airaksinen, M. Saarma, The GDNF family: signalling, biological functions and therapeutic value. Nat Rev Neurosci. 3 (2002) 383-394). It is also reported that intraspinal injection of GDNF into the injured spinal cord improved hindlimb recovery in rats with spinal cord injury (SCI) (H. Cheng, J. P. Wu, S. F. Tzeng, Neuroprotection of glial cell line-derived neurotrophic factor in damaged spinal cords following contusive injury. J. Neurosci. Res. 69 (2002) 397-405). According to the invention, treatment with GDNF improves the extension of neuritis and the extended processes with branching. In the aspect of neuron-specific markers, GDNF has a stimulatory effect on the expressions of neurofilament light protein (NF-L), vesicle protein-synapsin-1 and neuronal progenitor marker-internexin. GDNF also induces the neuronal nonspecific cytoskeleton protein α-tubulin expression in SSCs. Preferably, the dosage of glial cell line-derived neurotrophic factor is from 20 ng/mL to 50 ng/mL.

Pituitary adenylate cyclase-activating polypeptide (PACAP), a cAMP-inducing neuropeptide, has a critical role in CNS neural differentiation in vivo and in vitro (E. Dicicco-Bloom, N. Lu, J. E. Pintar, J. Zhang, The PACAP ligand/receptor system regulates cerebral cortical neurogenesis, Ann. N.Y. Acad. Sci. 11 (1998) 274-289; J. A. Waschek, Multiple actions of pituitary adenylyl cyclase activating peptide in nervous system development and regeneration, Dev. Neurosci. 24 (2002)14-23). Moreover, elevated intracellular cAMP is decisive for axonal regeneration (W. D. Snider, F. Q. Zhou, J. Zhong, A. Markus, Signaling the pathway to regeneration, Neuron. 35 (2002) 13-16). Gattei et al (V. Gattei, A. Celetti, A. Cerrato, M. Degan, A. De luliis, F. M. Rossi, G. Chiappetta, C. Consales, S. Improta, V. Zagonel, D. Aldinucci, V. Agosti, M. Santoro, G. Vecchio, A. Pinto, M. Grieco, Expression of the RET receptor tyrosine kinase and GDNFR-alpha in normal and leukemic human hematopoietic cells and stromal cells of the bone marrow microenvironment, Blood. 89 (1997) 2925-2937) have found that GDNF receptor-α and its accessory tyrosine kinase-RET were expressed in hBMSCs. According to the invention, PACAP is used to stimulate neuronal differentiation of the SSCs on the morphological transformation of the SSCs into neurons. PACAP stimulates neurogenesis via elevating intracellular cAMP through the PAC1 receptor (Dicicco-Bloom et al. (1998)). According to the invention, treatment with PACAP improves the extension of neuritis and the extended processes with branching. In the aspect of neuron-specific markers, PACAP has a stimulatory effect on the expressions of NF-L, vesicle protein-synapsin-1 and neuronal progenitor marker-internexin. PACAP also induces α-tubulin expression in SSCs. Preferably, the dosage of pituitary adenylate cyclase-activating polypeptide is about 10 ng/mL to about 20 ng/mL.

Dibutyryl cAMP (dbcAMP) is a cell permeable cAMP analog which induces highly branched, elongated, and delicate processes in SSCs. According to the invention, treatment with dbcAMP increases the expressions of NF-L, resicle protein-synapsin-1 and neuronal progenitor marker-internexin. dbcAMP also induces α-tubulin expression in SSCs. Furthermore, treatment with dbcAMP caused more extensive branched, elongated processes than those observed in GDNF- and PACAP-treated SSC cultures. Preferably, the dosage of dibutyryl cAMP is 100 μM.

Neurotrophic factors have effect on neuronal survival and repair for many neurological diseases. Thus, the combination of cell transplantation with neurotrophic factors is thought to be a potent therapeutic strategy for neurological diseases. It is evidenced by neural stem cell differentiation induced by neurotrophic factors (N.Y. Ip, The neurotrophins and neuropoietic cytokines: two families of growth factors actingon neural and hematopoietic cells, Ann. N.Y. Acad. Sci. 840 (1998) 97-106; A. Markus, T. D. Patel, W. D. Snider, Neurotrophic factors and axonal growth, Curr. Opin. Neurobiol. 12 (2002) 523-531; H. Thoenen, Neurotrophins and neuronal plasticity, Science. 270 (1995) 593-598). However, the effect of neurotrophic factors on trans-differentiation of SSCs into neuronal cells is unknown. The invention is the first to show that GDNF and PACAP can stimulate SSC differentiation toward a mature neuronal phenotype. The two neurotrophic factors are known to exert neuroprotection and to stimulate axonal regrowth via activating cAMP/PKA, MAP kinase, P13 kinase, and PLC-γ signaling pathways (Airaksinen et al. (2002) and Waschek et al. (2002)). In addition, elevating intracellular cAMP can enhance the formation of neurites in SSCs. The present invention showed that SSCs cultured in ITS medium have a process-bearing form and are positive for neuronal synapsin vesicle protein-synapsin-1. Treatment with GDNF or PACAP in ITS medium can further induce the transformation of SSCs into neuronal-like cells with dedicated processes. NF proteins expressed in most CNS mature neurons are known to play a crucial role in neuronal growth, organization, shape, and plasticity. Therefore, the additive expression of NF-L in SSCs induced by ITS medium containing GDNF or PACAP indicates the regulatory role of the two molecules on neuronal differentiation of SSCs. Extensive branching in elongated processes and increased levels of NF-L are observed in dbcAMP-treated SSCs, suggesting that intracellular cAMP may be involved in promoting neuronal differentiation of PACAP-treated SSCs. Note that no synergetic effect of GDNF combines with either PACAP or dbcAMP in the production of NF-L and α-tubulin in SSCs.

The following Examples are given for the purpose of illustration only and are not intended to limit the scope of the present invention.

### EXAMPLE 1

### Size-Sieved Stem Cell Derived from Human Bone Marrow

SSCs were isolated from human bone marrow as described previously (Hung et al. (2002)). In brief, human bone marrow was aspirated from the iliac crest of normal donors, and then washed twice with phosphate-buffered saline (PBS). The cells were loaded into 1.073 g/ml Percoll solution (Sigma®), and then centrifuged at 900 g for 30 min. The mononuclear cells (MNCs) were collected from the interface, and plated at the density of 1×10⁶ MNCs/cm² onto a 10-cm plastic culture dish comprised of an inserted sieve with 3-μm pores (Transwell System, Corning®). The cells were cultured in Dulbecco's modified Eagle's medium-low glucose (DMFM-LG) containing 10% fetal bovine serum (FBS), 100 U/ml penicillin, 100 mg/ml streptomycin, and 0.25 μg/ml amphotericin B (serum-containing medium). After 7 days, the cells adhering to the upper part of the inserted sieve had a larger, fibroblastic-like morphology, and were named SSCs. However, the cells that passed through the sieve were small, polygonal and had less renewal. SSCs were then harvested with 0.25% trypsin and 1 mM EDTA, and replated on 10-cm culture petri dishes. When SSCs were grown in serum containing medium to 80% confluence, the cells were replated onto 35-mm culture petri dishes at the density of 1×10⁵ cells/dish for Western Blotting, or onto 8-well chambers at the density of 1×10⁴ cells/well for immunofluorescence.

### EXAMPLE 2

### Stimulation of Size-Sieved Stem Cell Derived from Human Bone Marrow

After 48 h in serum containing medium, SSCs were treated in serum medium (ITS medium) alone, and in ITS medium containing GDNF (20 and 50 ng/ml, R&D®), PACAP (10 and 20 ng/ml; Sigma®), or dbcAMP (20 μM; Sigma®). ITS medium consisted of 56% DMEM-LG (Life Biotech®), 40% MCDB-201 medium (Sigma®), and 1×ITS medium supplement (Sigma®) contained 1 mg/ml insulin, 0.55 mg/ml human transferrin, 0.5 μg/ml sodium selenite, 10 nM dexamethasone (Sigma®), and 10 μM ascorbic acid (Sigma®)). We found that SSCs in DMEM-LG medium without serum were founded to respond poorly to GDNF and PACAP. However, when SSCs were cultured in serum free medium with ITS supplement, these cells adhered well onto cultured plates and extended their processes. Therefore, treatments were performed in ITS medium.

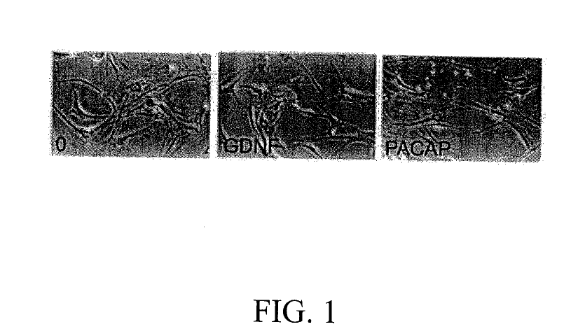

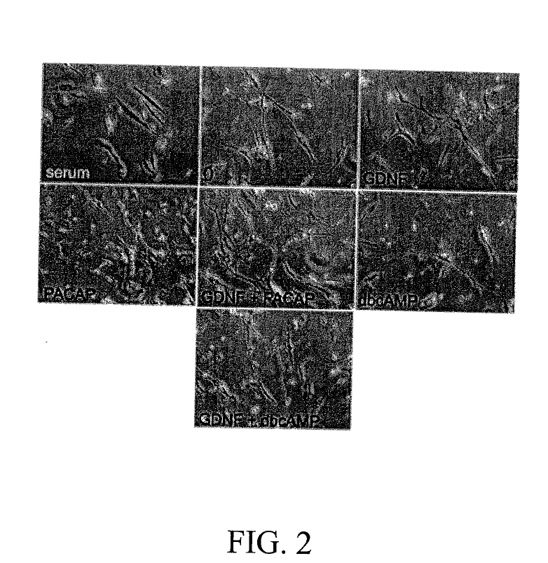

The morphology of SSCs is as shown in FIGS. 1 and 2. As shown in FIG. 2, when SSCs were cultured in 10% FBS-containing medium, the cells had a flat, fibroblastic-like morphology. However, SSCs were found to generate extended neurites while SSCs were cultured in ITS medium alone (FIGS. 1 and 2). In addition, treatment with SSCs in ITS medium containing GDNF or PACAP further improved the extension of neurites. It indicated that the vesicle protein, synapsin-1, was already observed when SSCs were cultured for 7 days in ITS medium alone. Given the above, ITS medium alone also induced the neuronal differentiation of SSCs to some extent.

### EXAMPLE 3

### Effects of GDNF and PACAP and dbcAMP on Neuron-Specific Markers

This is to study the effects of GDNF, PACAP and dbcAMP on neuronal differentiation of SSC in ITS medium. The levels of neuron-specific markers (NF-L and NF-H) in the SSCs were examined by Western Blotting. The cells were replated at the density of 1×10⁵ cells per 35 mm petri dish and cultured for 7 days in ITS medium with GDNF, PACAP or dbcAMP at the distinct concentrations. Cells were harvested and gently homogenized on ice using PBS containing 1% SDS, 1 mM phenylmethyl-sulfonylfluoride (PMSF), 1 mM EDTA, 1.5 mM pepstatin, 2 mM leupeptin, and 0.7 mM aprotinin. Protein concentrations were determined using a Bio-Rad® DC kit. Ten μg of total protein was loaded onto 7.5% SDS-PAGE, and transferred to a nitrocellulous membrane. NF-L (70 kDa) and NF-H (200 kDa) were identified by incubating the membrane with anti-NF antibodies (Chemicon®) overnight at 4° C., followed by horseradish peroxidase-conjugated secondary antibodies and enhanced chemiluminescence solution (NEN LifeScience®).

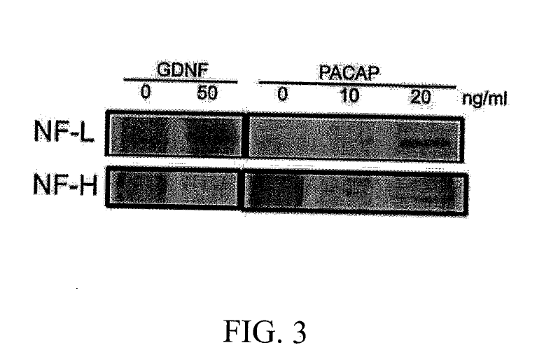

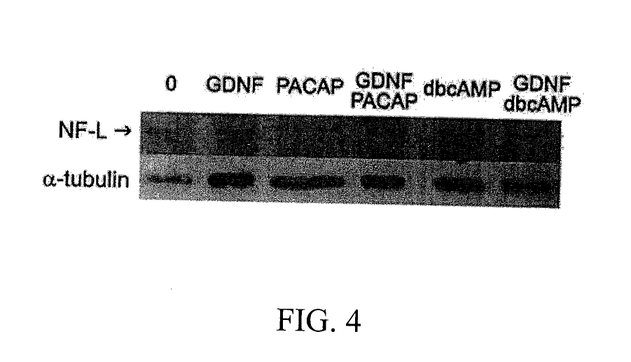

The results are shown in FIGS. 3 and 4. Referring to FIG. 3, Western Blotting showed that treatment with GDNF or PACAP had a stimulatory effect on the expression of NF-L protein in SSCs, albeit less effect on NF-H levels in SSCs.

As shown in FIG. 4, ITS medium containing the cell permeable cAMP analog, dbcAMP, induced highly branched, elongated, and delicate processes in SSCs when compared to those observed in ITS medium alone. As with GDNF and PCACP, Western Blotting also indicated that treatment with dbcAMP increased the level of NF-L and α-tubulin in SSCs. Note that there was no synergetic effect of GDNF combined with either PACAP or dbcAMP on the production of NF-L and α-tubulin in SSCs.

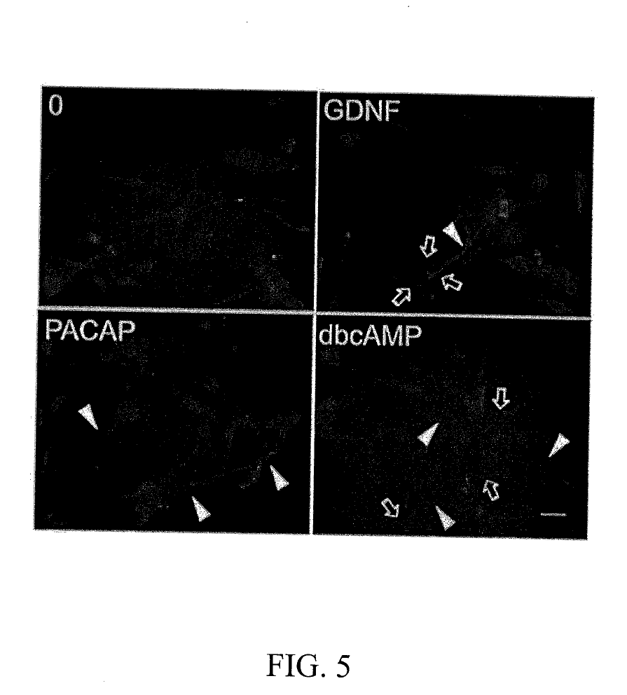

Immunofluorescence staining for another neuronal intermediate filament protein, α-internexin (FIG. 5), was performed using antibodies against α-internexin (1:200; Chemicon®). It evidenced that GDNF and PACAP could induce process branching of SSCs to some extent. However, treatment with dbcAMP for 7 days caused more extensive branched, elongated processes than those observed in GDNF- and PACAP-treated SSC cultures, as well as control cultures.

### EXAMPLE 4

### Effects of GDNF and PACAP on Morphological Change of SSCs

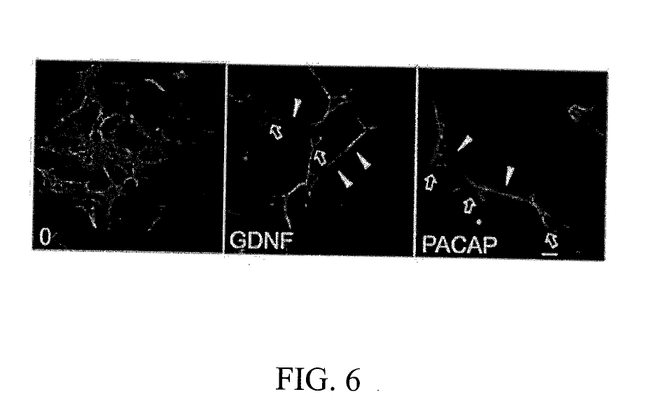

To detect the morphological change of the SSCs that had been treated with 50 ng/ml GDNF or 20 nM PACAP in ITS medium for 7 days, the SSCs were fixed in PBS-containing 4% paraformaldehyde for 10 minutes. Examination of immunofluorescence for synapsin-1, a synapse vesicle protein, was performed by incubating SSCs with anti-synapsin-1 antibodies (1:200; BD Biosciences®) in PBS-containing 0.1% Triton X-100 and 5% horse serum overnight at 4° C., followed by biotinylated secondary antibodies (1:200; Vector®) and FITC-avidin (1:200; Vector®). The results indicated that a strong immunofluorescence staining for synapsin-1 was observed in the processes and/or peripheral membranes of SSCs with or without treatment. Yet, it was evident that synapsin-1 positive cells in GDNF- or PACAP-treated cultures showed extended processes with branching (as shown in FIG. 6).

### EXAMPLE 7

### Study of Hindlimb Locomotor Recovery after SSCs Transplantation

The in vitro studies described above suggested that SSCs could be a potential cell source for cell-based therapy in combination with PACAP and GDNF for spinal cord injury (SCI) treatment. To confirm this, experiments were performed to determine delayed transplantation of SSCs (80,000-200,000 SSCs/rat) with PACAP (2 μg/rat) into injured spinal cord of rats at 8 days post contusive injury (severe SCI), based on whether and the extent of this cell/factor-based treatment can improve hindlimb locomotor recovery in severe SCI rats.

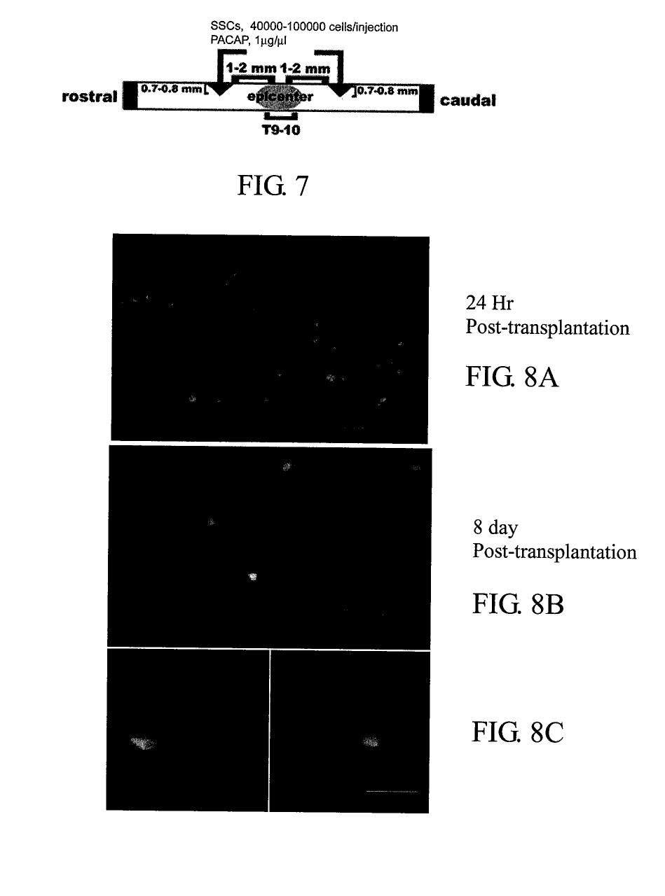

SCI was induced using a weight-drop device developed at New York University (S. Constantini and W. Young, “The effects of methylprednisolone and the ganglioside GM1 on acute spinal cord injury in rats,” J. Neurosurg. 80 (1994), 97-111; D. M. Basso, M. S. Beattie, J. C. Bresnahan. “Graded histological and locomotor outcomes after spinal cord contusion using the NYU weight drop device versus transection.” Exp. Neurol. 139 (1996) 244-256). The animal surgical procedure was performed as reported previously (Cheng et al., 2002; Tai et al., 2003; Chen and Tzeng, 2005). Briefly, adult female Sprague-Dawley rats (250±20 g) were anesthetized with pentobarbital (50 mg/kg), a laminectomy was performed at T9-T10, and the dorsal surface of the spinal cord was compressed by dropping a 10-gm rod from a height of 50 mm, resulting in severe SCI. At 7-8 days after contusive injury, the rats received SSCs+PACAP transplantation. A 10-μl Exmire microsyringe with a 31-gauge needle was positioned at the midline of the cords 2 mm rostral and caudal (dual injection) to the contusive center. SSC cells (4-10×10⁴ cells/injection; less than 2×10⁵ cells/animal) were injected 0.8 mm into the dorsal column of the spinal cord. This is diagrammed in FIG. 7. The transplanted SSCs were maintained in ITS medium containing PACAP (20 ng/ml) for 2-3 days before transplantation. Less than 10 μl total volume of cells plus PACAP (1μ/injection) were used for one injection. Each injection was completed within 10 min. Bladder evacuation was applied daily for at least 7 days. Antibiotics (sodium ampicillin, 80 mg/kg) were injected daily into animals for 7-9 days. All surgical interventions and animal care was performed in accordance with the Laboratory Animal Welfare Act, the Guide for the Care and Use of Laboratory Animals, and National Cheng Kung University.

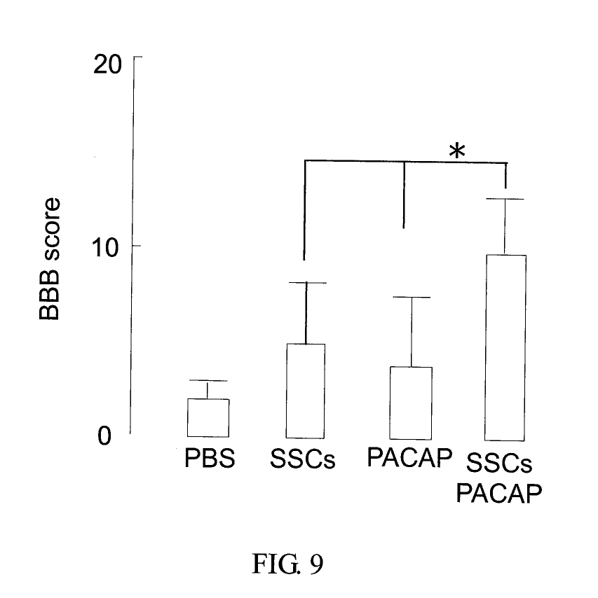

Immunostaining for the specific marker (Hu) of SSCs showed that Hu⁺ cells at 1 day (FIG. 8A) and 8 days (FIG. 8b) post-SCI existed in the injected site proximal to the epicenter (FIG. 8C). The Basso Beattie Bresnahan (BBB) locomotor rating analysis (Brasso et al. 1996) was performed 4 weeks after SCI. The results are graphed in FIG. 9, and indicated that delayed transplantation of SSCs/PACAP increased hindlimb locomotion in rats that had severe SCI in the form of severe spinal cord contusion.

While embodiments of the present invention have been illustrated and described, various modifications and improvements can be made by persons skilled in the art. It is intended that the present invention is not limited to the particular forms as illustrated, and that all the modifications not departing from the spirit and scope of the present invention are within the scope as defined in the appended claims.

We claim:

1. A method for treating neural injury or improving the recovery of spinal injury, the method comprising providing sieve-sized stem cells cultured with at least one of dibutyryl cAMP (dbcAMP) and a neurotrophic factor or a combination thereof, and transplanting to the neural or spinal injury the sieve-sized stem cells with the dbcAMP, the neurotrophic factor or a combination thereof.

2. The method of claim 1, wherein the neurotrophic factor comprises glial cell line-derived neurotrophic factor (GDNF) or pituitary adenylate cyclase-activating polypeptide (PACAP).

3. The method of claim 2, wherein the neurotrophic factor is PACAP.

4. The method according to claim 1, wherein the bone marrow stem cells are size-sieved stem cells derived from human bone marrow.

5. The method according to claim 4, wherein the size-sieved stem cell is sieved with a
3-μm porous sieve.

6. The method according to claim 2, wherein the size-sieved stem cell is sieved with a 3-μm porous sieve.

7. The method according to claim 3, wherein the PACAP is present in a dosage of about 10 ng/mL to about 20 ng/mL.

8. The method according to claim 1, wherein the dosage of dibutyryl cAMP is 100 μM.

9. A method for preparing a bone marrow stem cell for induction of neural differentiation, the method comprising treating a size-sieved human bone marrow stem cell with pituitary adenylate cyclase-activating polypeptide (PACAP).

10. The method according to claim 9, wherein the neural differentiation comprises one or more changes selected from the group consisting of neurofilament light protein (NF-L) increasing, α-tubulin increasing, vesicle protein-synapsin-1 production, neuronal progenitor marker-internexin production, cell processes elongation, and process branching increasing.

11. The method according to claim 2, wherein the size-sieved stem cell is sieved with a 3-μm porous sieve.

12. The method according to claim 3, wherein the PACAP is present in a dosage of about 10 ng/mL to about 20 ng/mL.

13. The method of claim 9, further comprising applying the size-sieved stem cell treated with PACAP to a site in need of neural differentiation.
